(19) United States
(12) Patent Application Publication
(10) Pub. No.: US 2016/0242872 A1
(43) Pub. Date: Aug. 25, 2016

(54) ORAL INSERT

(71) Applicant: Michael STEFFLING, Chian Mai (TH)

(72) Inventor: Michael Steffling, Chian Mai (TH)

(21) Appl. No.: 14/915,566

(22) PCT Filed: Aug. 30, 2013
(86) PCT No.: PCT/CH2013/000154

§ 371 Date: Feb. 29, 2016

(30) Foreign Application Priority Data
Aug. 31, 2012 (CH) 1560/12

(51) Int. Cl. A61C 7/12; A61M 16/04; A61M 16/10; A61F 5/56

(52) U.S. Cl. CPC A61C 7/125; A61F 5/566; A61M 16/0493; A61M 16/106; A61M 2205/0216

(57) ABSTRACT

The invention relates to an oral insert for positioning between the outer faces of the teeth and the lips of a user, comprising a strip having a length and a width and an inner and outer face, wherein in the region of the longitudinal centre the longitudinal sides have an indentation. The oral insert further comprises at least one respiratory opening penetrating through the strip and a first and second bite projection which protrude from the inner face of the strip. The at least one respiratory opening is integrated in the first or second bite projection.

### CROSS-REFERENCE TO RELATED APPLICATIONS

This application is a national phase entry under 35 U.S.C. §371 of PCT/CH2013/000154 filed on Aug. 30, 2013, which claims priority to Swiss Patent Application 1560/12 filed on Aug. 31, 2012, the entirety of each of which is incorporated by this reference.

### FIELD OF THE INVENTION

The invention relates to an oral insert for positioning between the outer faces of the teeth and the lips of a wearer.

### PRIOR ART

A patient wearing orthodontic braces suffers from pain on the inside of the lips. This is because the braces are raised and usually have sharp edges against which the sensitive oral mucosa rubs. The wearing of orthodontic braces and the pressure which is necessarily present on the teeth leads to a tensioning of the mouth and of the facial musculature which also results in pain.

U.S. Pat. No. 5,447,168 discloses a lip protection in the form of a thin, curved strip. The strip is positioned on the outer face of the teeth between the teeth and the lips to shield the lips from the outside of the teeth and from all the elements present there of an orthodontic brace. The length is selected such that all the teeth of a normal set of teeth are covered on both sides from the incisors to the molars. The height of the strip is selected such that the upper teeth and lower teeth are covered. Continuous openings which facilitate breathing are positioned centrally both with respect to the length and to the height of the strip, in the region of the incisors. Located in each case at the side of these respiratory openings in the direction of the molars are small bite bars which are approximately in the middle with respect to the height of the strip and project vertically from the strip. The bite bars can be engaged with the molars and are to prevent the patient from grinding his teeth and to prevent muscle tension in the mandibular joint. However, the bite bars cannot be adapted to the wearer's teeth and the wearer can only breathe through the respiratory openings when his mouth is open.

### ADVANTAGE OF THE INVENTION

It is therefore an advantage of the invention to propose an improved oral insert.

### SUMMARY OF THE INVENTION

According to the invention, the advantage is achieved with an oral insert for positioning between the outer faces of the teeth and the lips of a wearer comprising a strip having a length and width and an inner and outer face, wherein the longitudinal sides have an indentation in the region of the longitudinal center, at least one respiratory opening passing through the strip and first and second bite projections that protrude from the inner face of the strip and wherein the at least one respiratory opening is integrated into the first or second bite projection. This constructional feature makes it possible to arrange the respiratory opening and the bite projection in a common desired area of the strip. It is therefore possible to dispense with the separate provision of at least one respiratory opening. This is particularly practical when the wearer himself determines the position of the bite projection. Only one passage opening then needs to be cut into the strip per bite projection with respiratory opening. The joint cleaning of bite projection and respiratory opening is also facilitated. It is advantageous if the first and second bite projections have an integrated respiratory opening. The bite projection can be configured as a web, a bar or as a cylindrical or conical projection. It is also possible for a breathing air filter to be integrated into the respiratory opening. Consequently, the filter function which is present during nasal breathing can also be provided during mouth breathing with the oral insert.

In one embodiment, the respiratory opening is realised by a tube piece which is guided through the first and/or second bite projection. The tube piece stabilises the bite projection and serves as a lug to fasten the bite projection to the strip. The tube piece is produced from a solid plastics material which is easy to clean and as far as possible does not allow bacteria to build up thereon.

The tube piece on the outer face of the strip expediently projects beyond said strip by at least 10 mm, by 15 mm or by 20 mm. This linear dimension makes it possible for the wearer to be able to breathe through his mouth even when his lips are closed. If this is not desired, the tube pieces can be shortened at any time.

In addition to the use of the oral insert when orthodontic braces or brackets are worn, the oral insert can also be use as a respiratory training device. The mouth of a person who is asleep is closed in most cases and the sleeping person breathes through his nose. This can have a negative impact on the entire respiratory system during sleep. The oral insert ensures that sufficient air is supplied through the oral respiratory tract during sleep and the bite knobs fix the jaw musculature. During sleep, due to the oral insert, the respiratory cycle of the oral tract and nasal passages remains intact and the entire spinal system is relieved due to the fixing of the jaw musculature.

Since, in one embodiment, the tube piece has a first, second and third notch, the strip, the retaining ring and the bite knob can be fixed positively on the tube piece. The individual parts form a stable unit, but can be separated at any time from one another by being pulled off from the tube piece. This function is particularly practical, especially for cleaning purposes or if one of the parts is broken.

It proves to be advantageous when the strip can be fixed on the tube piece by frictional locking and/by form-locking. As stated in the last paragraph, the individual parts can be quickly interconnected due to this constructional feature, for example after the strip has been adapted to the respective size of the wearer's mouth. The individual parts can also be separated again just as quickly (for example, for cleaning).

It proves to be advantageous when a retaining ring can be pushed onto the tube piece, which retaining ring, when mounted, rests on the outer face of the strip. It is therefore very simple to attach the bite projection together with the tube piece by pushing the retaining ring onto the projecting tube piece.

The retaining ring can be fixed on the tube piece by friction locking and/or by form locking. It is understood that for an effective retaining function, the retaining ring is produced from a flexible material, for example from rubber. Furthermore, the internal diameter of the retaining ring should be slightly smaller than the external diameter of the tube piece. To improve the retaining function, it is possible for a peripheral notch to be provided on the tube piece in front of the outer face of the strip. The retaining ring engages in this notch and is thus also held in a form-locking manner on the tube piece.

In a further embodiment, the first and second bite projections are bite knobs which are substantially cylindrical. The cylindrical shape of the bite knob allows a comfortable retention of the oral insert by the teeth. In particular, when the bite knobs are arranged between teeth 3 and 4 of the dentition quadrants, the knobs are in a particularly comfortable position for the wearer. However, as stated below, it is also possible to position the bite knobs in any other area between the teeth of the upper jaw and lower jaw.

The bite knob expediently has on its end remote from the inner face a front plate, the diameter of which is greater than the diameter of the rest of the bite knob. The front plate allows the wearer to fix the oral insert more effectively to the teeth and to substantially prevent the oral insert from slipping.

The bite knob advantageously has an outer part which is produced from a soft, flexible material, in particular from a rubber. This material allows the oral insert to be held in a comfortable manner, because the bite knobs deform in response to the pressure of the teeth between which they are held.

In another embodiment, the bite knob can be clipped into the strip. As described above, this can be facilitated by the respiratory tube and retaining ring. It is also possible for the tube piece to have a notch which can be clipped into a passage opening in the strip.

Due to the fact that the outer contour of the strip is provided with an annular bead, the oral insert does not have any sharp edges and is pleasant to wear. To provide comfort during wear, the annular bead is produced from a soft flexible material, for example from silicone.

To be able to effectively clean the annular bead and the transition between annular bead and strip, it is advantageous if the annular bead can be attached to the strip such that it runs around the edge thereof.

In a further embodiment, cutting lines using which the strip can be cut to different sizes are provided in the region of the outer contour. Since the strip can be cut to size, it can be adapted to the individual anatomical requirements of the wearer. The cutting lines indicate typical sizes on the strip, as a result of which the wearer himself can cut out an S, M, L or XL oral insert.

A further aspect of the invention relates to a multi-part set comprising the strip, the bite knobs and a rubber bead. Due to the fact that passage openings can be cut into the strip, the bite knobs can be fastened anywhere on the strip. The cutting lines for the passage openings can already be marked on the strip. This is practical in that the passage openings have the correct diameter for the bite knobs. The passage openings or the cutting lines for the passage openings are arranged symmetrically to the width centre line and on the longitudinal centre line of the strip.

Further advantages and features will be revealed from the following description of an embodiment of the invention with reference to the schematic drawings which are not true-to-scale.

### BRIEF DESCRIPTION OF THE DRAWINGS

FIG. 1 is a side view of the oral insert according to the invention;

FIG. 2 is a sectional view through the oral insert form FIG. 1 in the region of a bite knob;

FIG. 3 shows a section through a tube piece, and

FIG. 4 is a perspective diagonal top view of the oral insert including two retaining rings.

### DETAILED DESCRIPTION OF THE INVENTION

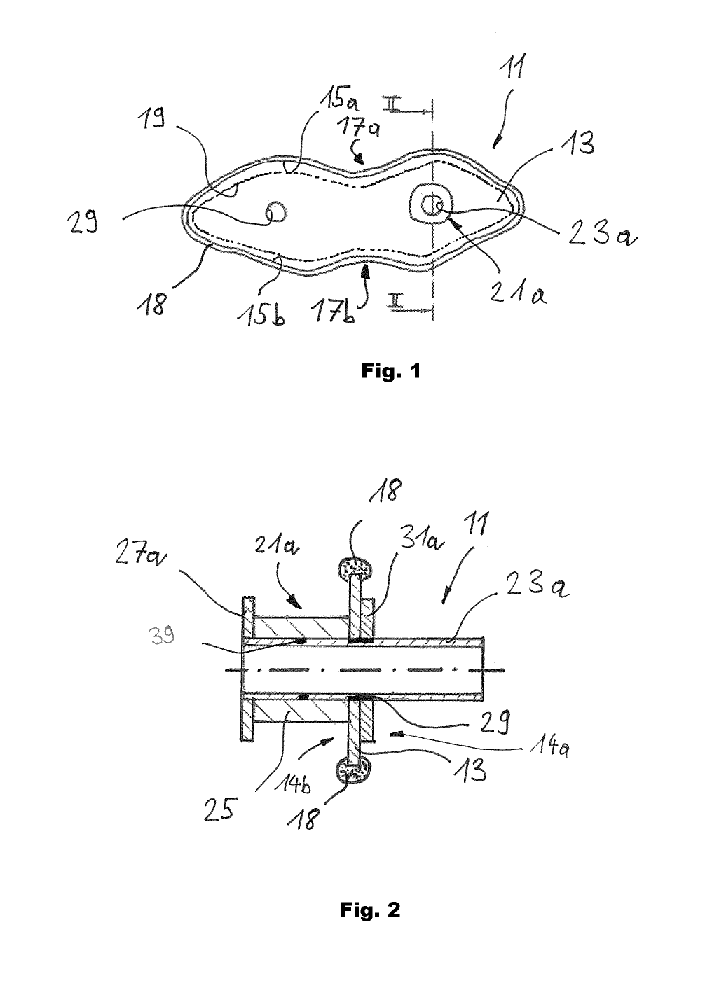

FIGS. 1, 2 and 4 show the oral insert according to the invention denoted overall by reference numeral 11. The oral insert 11 comprises as a base part a strip 13 having an outer face 14a which, when the insert is worn, faces the wearer's lips, and an inner face 14b which faces the wearer's teeth. Therefore, the oral insert 11 is to be positioned inside the mouth between the teeth and the lips. The length of the strip 13 is selected such that it covers the entire dental arch of an adult. The height is selected such that the teeth of the upper jaw and of the lower jaw are covered. The strip is produced from a flexible, washable and water-tight film material. Silicone materials or rubber materials are possible, as is plastics-coated cardboard or the like. Since the strip has to be pliable and flexible so that it can be adapted to the dental arch or lips of the wearer, it has layer thicknesses of at most 1 mm.

In the region of the line between the chin and nose of the wearer, the longitudinal sides 15a, 15b of the strip 13 have a respective indentation 17a, 17b to make the oral insert 11 more comfortable to wear. The width of the strip 13 decreases again in the direction of the molars. The lateral ends of the strip 13 are rounded. To further improve the wearer's comfort, the outer contour of the strip 13 is bordered by an annular bead 18. The annular bead 18 is slotted on the inside and consequently can be easily arranged along the outer contour of the strip 13. The annular bead 18 can also be removed for cleaning purposes. The bead 18 is produced from a flexible material which is kind to the skin, for example silicone. In order that the annular bead 18 can be adapted particularly effectively to the contours of the strip 13, it can also be an open annular bead strip, or it can be composed of a plurality of annular bead strips. These annular bead strips are not tensioned and conform exactly to the outer contours or curves of the strip. The annular bead strip or strips can be individually adapted to the length of the outer contour of the strip 13 by trimming.

It is provided that the wearer of the oral insert 11 adapts the length and width of the insert 11 to his requirements by cutting off the side edges as required. For this purpose, one or more cutting lines 19 can be marked on the strip 13 in the region of its outer contour. Cutting lines 19 of this type are known from insoles which can be adapted to different shoe sizes.

A first and second bite knob 21a, 21b can be attached to the strip 13. A respective tube piece 23 is guided through the bite knobs 21a, 21b. The length of the tube piece portion which projects beyond the outer face of the strip 13 is selected such that even when the lips are closed, the wearer can breathe in and out through the tube pieces 23a, 23b. As shown in FIG. 2, the bite knob 21a and the tube piece 23a are arranged substantially vertically to the strip 13. FIG. 1 shows that it is advantageous when the bite knobs 21a, 21b are arranged symmetrically to the longitudinal and width centre lines. Adjoining the inner face of the strip 13, the tube piece 23 is surrounded by a centre portion 25a, 25b which consists of a soft, flexible material, also silicone or rubber. To facilitate the retention on the teeth, the free end face of the bite knob 21a, 21b is terminated by a front plate 27a, 27b. The diameter of the front plate 27a, 27b is slightly greater than the diameter of the centre portion 25a, 25b.

Provided in the strip 13 are passage openings 29 through which the tube pieces 23a, 23b can be guided. In this respect, the internal diameter of the passage openings 29 substantially corresponds to the external diameter of the tube piece 23a, 23b. The passage openings 29 can already be provided during the production of the strip 13 or they are cut out in the places required by the wearer. The passage openings 29 are expediently cut out in one area of the strip 13 so that, during use, they are positioned between teeth 3 and 4 of the dentition quadrants. However, the wearer can be flexible when arranging the bite knobs 23a, 23b. To fix the bite knobs 23a, 23b on the strip 13, retaining rings 31a, 31b can be pushed onto the tube pieces 23a, 23b which project beyond the outer face. To ensure a friction-locking mount of the retaining rings 31a, 31b, said retaining rings are produced from a flexible material, in particular from rubber or silicone.

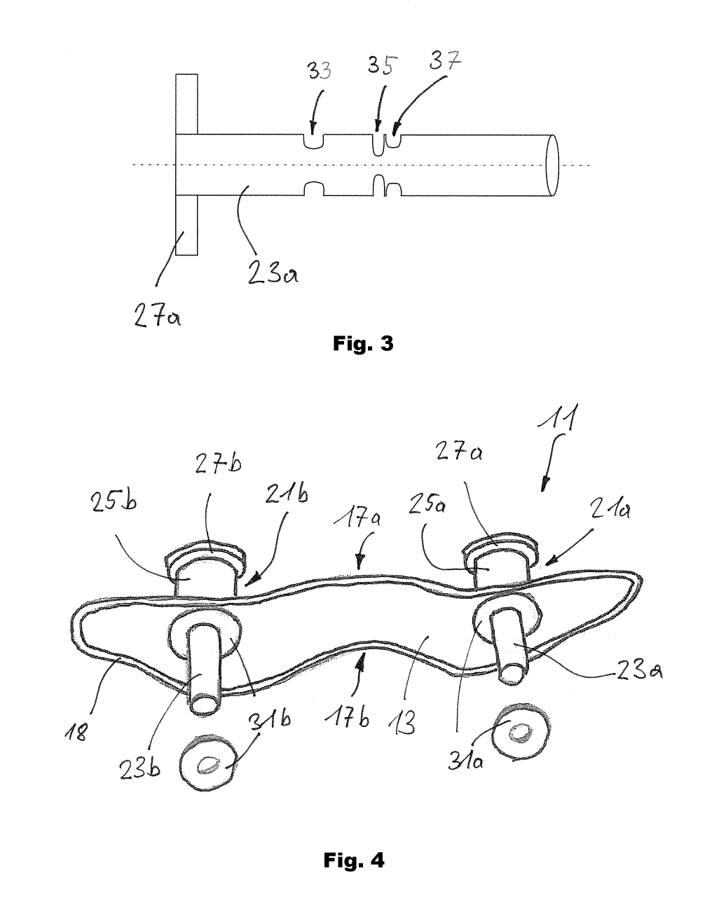

FIGS. 2 and 3 show that the tube pieces 23a, 23b have a first, second and third notch 33, 35, 37. The first notch 33 is used to hold the centre portion 25a, 25b in a positive manner. For this purpose, the centre portion 25a, 25b can have projections 39 which can be latched into the first notch 33. The second notch 35 is used to positively receive the strip 13. In this respect, the passage opening 29 in the strip 13 is dimensioned such that it can latch into the second notch 35. Similarly, the retaining ring 31a, 31b can be latched into the third notch 37. The notches 33, 35, 37 must be present at least in one place on the surface of the tube piece 23a, 23b for a positive locking. It is understood that the notches 33, 35, 37 can also run around the periphery of the tube piece 23a, 23b. It is also possible to dispense with the third notch 37 and the retaining ring, since the strip 13 is held firmly enough in notch 35.

For cleaning purposes, the bite knobs 21a, 21b can be separated quickly from the strip 13 by pulling off the retaining rings 31a, 31b and after cleaning; they can also be quickly re-inserted into the strip 13.

The oral insert 11 reliably protects the inside of the lips against brackets and allows the wearer to breathe even when the lips are closed. Furthermore, wearing the oral insert relaxes the lips and the facial musculature. The integral construction of tube piece 23a, 23b and bite knobs 21a, 21b makes it possible to quickly attach this unit to the strip 13 and to quickly remove it therefrom. The oral insert 11 can be adapted very effectively to the wearer's anatomical requirements: on the one hand, the strip 13 can be cut to size. On the other hand, the bite knobs 21a, 21b can be positioned where desired on the strip 13, since the passage openings 29 can be cut out anywhere from the strip 13.

1-16. (canceled)

17. Oral insert for positioning between the outer faces of the teeth and the lips of a wearer, comprising:
a strip having a length and width and an inner and outer face, wherein the longitudinal sides have an indentation in the region of the longitudinal center, at least one respiratory opening passing through the strip and first and second bite projections that protrude from the inner face of the strip, the at least one respiratory opening being integrated into at least one of the first or second bite projection.

18. The oral insert according to claim 17, wherein the respiratory opening is comprised of a tube piece that is guided through at least one of the first and second bite projection.

19. The oral insert according to claim 18, wherein the tube piece on the outer face of the strip projects beyond said strip by at least 10 mm.

20. The oral insert according to claim 18, wherein the tube piece includes a first, second and third notch.

21. The oral insert according to claim 18, wherein the strip can be fixed on the tube piece in at least one of a friction locking or form locking manner.

22. The oral insert according to claim 18, further comprising a retaining ring pushed onto the tube piece, the retaining ring, when mounted, rests against the outer face of the strip.

23. The oral insert according to claim 18, further comprising a retaining ring fixed on the tube piece in at least one of a friction locking or form locking manner.

24. The oral insert according of claim 17, wherein the first and second bite projections comprise first and second bite knobs, respectively, that are substantially cylindrical.

25. The oral insert according to claim 24, wherein each of the first and second bite knobs have a first diameter on an end remote from the inner face a front plate that is greater than a second diameter of the rest of each of the first and second bite knobs.

26. The oral insert according to claim 24, each of the first and second bite knobs has an outer part that is produced from a soft, flexible material.

27. The oral insert according to claim 24, wherein the first and second bite knobs can be clipped into the strip.

28. The oral insert according to claim 17, wherein an outer contour of the strip includes an annular bead.

29. The oral insert according to claim 28, wherein the annular bead is attached to the strip so that it runs around an edge thereof.

30. The oral insert according to claim 28, wherein in a region of the outer contour are cutting lines configured to be used to cut the strip to different sizes.

31. The oral insert according to claim 17, further comprising a breathing air filter integrated into the respiratory opening.

32. A multi-part oral insert set, comprising:
a strip having a length and width and an inner and outer face, wherein the longitudinal sides have an indentation in the region of the longitudinal center, at least one respiratory opening passing through the strip and a first and second bite
projection that protrude from the inner face of the strip, the at least one respiratory opening being integrated into the first or second bite projection;
a plurality of passage openings formed in the strip for guiding the bite knobs through the strip; and
a rubber bead configured to be attached to an edge of the strip.
